(19) United States
(12) Patent Application Publication
(10) Pub. No.: US 2011/0219466 A1
(43) Pub. Date: Sep. 8, 2011

(54) SEQUENCE-CHARACTERIZED AMPLIFIED REGION (SCAR) MARKERS FOR PLANT RESISTANCE AGAINST FUNGAL PATHOGENS

(76) Inventors: Dilantha Fernando, Winnipeg (CA); Mathieu Dusabenyagasani, Winnipeg (CA)

(21) Appl. No.: 12/680,315

(22) PCT Filed: Oct. 3, 2008
(86) PCT No.: PCT/CA2008/001779

§ 371 Date: Oct. 21, 2010

Related U.S. Application Data
(60) Provisional application No. 60/977,933, filed on Oct. 5, 2007.
(60) Provisional application No. 61/021,529, filed on Jan. 16, 2008.

(51) Int. Cl. A01H 1/02; C07H 21/04; C12Q 1/68

(52) U.S. Cl. 800/265; 536/23.6; 435/6.11

(57) ABSTRACT

An isolated nucleic acid molecule comprising one of a nucleotide sequence set forth in SEQ ID NO: 1 and a nucleotide sequence set forth in SEQ ID NO: 2, the nucleotide sequence complementary to a Rpg3Dun gene sequence. A method for screening a plurality of plant cultivars to identify individual cultivars containing Rpg3Dun gene sequences, comprising the steps of: (a) collecting a biomass sample from individual cultivars; (b) preparing a nucleic acid preparation from each biomass sample; (c) contacting each nucleic acid preparation with at least one of the nucleic acid molecules set forth in SEQ ID NO: 1 and SEQ ID NO: 2, (d) performing a PCR amplification reaction therein; and (e) assessing each amplified sample to detect hybridization with one of the nucleic acid molecules set forth in SEQ ID NO: 1 and SEQ ID NO: 2. A hybridization reaction indicates the cultivar contains therein Rpg3Dun gene sequences.

### TECHNICAL FIELD

This invention relates to development of disease-resistant plant cultivars. More particularly, this invention relates to genetic markers and uses thereof for aiding the selection of disease-resistance progeny in breeding programs.

### BACKGROUND OF THE INVENTION

Blackleg (Phoma stem canker) of rapeseed and canola (Brassica napus L.) is a disease with a worldwide distribution caused by a complex of fungal plant pathogens belonging to Leptosphaeria maculans (Desm.) Ces. & Not., anamorph Phoma lingam (Tode ex Fr.) Desm. Members of this species used to be divided into two pathotype groups, A and B (also A and NA groups, or Tox⁺ and Tox⁰ groups), based on characters such as virulence, RFLP markers, and secretion of the phytotoxin sirodesmin into culture media. Group A isolates are highly virulent and cause the damaging stem canker while B group isolates are weakly virulent (Rouxel et al., 2004, IN Plant Genome: Biodiversity and Evolution, vol. 2: Lower Groups, Sharma, A. K., and Sharma, A., Eds. Enfield: Science Publishers, Inc., pp. 33-'7547; Williams et al., 1999, Plant Pathol. 48:161-175.). Since group B isolates are morphologically different from and cannot sexually cross with group A isolates, they have been recently classified as a new species, L. biglobosa (Shoemaker et al., 2001, Can. J. Bot. 79:412-419). Group A isolates are distributed worldwide and cause significant losses in Australia, Canada, and Europe (West et al., 2001, Plant Pathol. 50:10-27). They have been subdivided into pathogenicity groups (PG) 2, 3, 4 and T according to differential disease reactions on a set of Brassica napus varieties (lines) with different resistance genes (Koch et al., 1991, Mol. Plant-Microbe Interact. 4:341-349; Rimmer, 2006, Can. J. Plant Pathol. 28:S288-S297). The subspecies structure of L. maculans is made even more complex by the fact that each of the four pathogenicity groups also comprises different races as revealed by analysis conducted with two differential sets (Balesent, et al., 2005, Phytopathology 95:1061-1071).

Although restricted to the crucifers, L. maculans can infect various species under field and laboratory conditions. The host and the pathogen establish typical gene-for-gene interactions (Ferreira et al., 1995, Phytopathology 85:213-217; Pongam et al., 1998, Phytopathology 88:1068-1072; Yu et al., 2005, Theor. Appl. Genet. 110:969-979). The outcome of the host infection is dependent on the presence of a major resistance gene in the host and a corresponding avirulence gene in the pathogen (Ansan-Melayah et al., 1995, Phytopathology, 85:1525-1529; Ansan-Melayah et al., 1998, Plant Breed. 117:373-378; Balesdent et al., 2002, Phytopathology 92:1122-1133). Resistance genes have been traced in various Brassica species such as B. juncea, B. napus, B. rapa, and B. nigra (14,40,50,52). Selection for blackleg resistance relies on cotyledon stage greenhouse and adult stage field resistance (43). Several authors have reported a significant correlation between resistance in cotyledons and adult plants (8,22,25,34,37). Loci controlling cotyledon resistance as well as field resistance against L. maculans have been mapped to the N7 linkage group of the A genome in various B. napus (16,18,32,33,41,44). Molecular markers associated with Brassica resistance against blackleg caused by PG2 and useful for maker-assisted-selection have also been reported (Ananga, et al., 2006, Afr. J. Biotechnol. 5:2041-2048).

Blackleg of canola, which had been prevalent in the Canadian prairies for the last two decades, was caused by L. maculans PG2 and is well controlled by resistance. In 2002 and 2004, PG3 and PG4 isolates of L. maculans were discovered in Manitoba (Chen et al., Plant Disease 89: 339; Fernando et al., 2003, Plant Disease 87:1268). Since then, PG3 and PG4 have been also found in Alberta, N. Dak. (USA), and more regions in Manitoba (Chen et al., 2006, Can. J. Plant Pathol. 28:533-539). These new strains of L. maculans pose a serious threat to the Prairie canola industry because varieties currently grown are not resistant against them.

### SUMMARY OF THE INVENTION

The exemplary embodiments of the present invention are directed to isolated nucleotide sequences configured into a nucleic acid molecule that is complementary to the Rpg3Dun gene sequence, to methods for preparation of BN204, to nucleotide cassettes comprising BN204, to methods for preparing nucleotide cassettes comprising BN204, to kits comprising BN204 for using in Brassica progeny screening programs, and to screening methods for the use of BN204 to identify progeny individuals from Brassica breeding programs that contain therein putative gene sequence Rpg₃Dun.

According to one embodiment of the present inventions, there is disclosed a first SCAR marker complementary to the Rpg3Dun gene sequence, said SCAR marker isolated from Brassica napus F₂ ‘Westar’ and accessible with GenBank® accession number EU122190.

According to one embodiment of the present inventions, there is disclosed a SCAR marker complementary to the Rpg3Dun gene sequence, said SCAR marker isolated from Brassica napus ‘Dunkeld’ and accessible with GenBank® accession number EU122191.

For clarity, the term “BSA” as used herein refers to bulked segregant analysis.

The term “SRAP” as used herein refers to sequence-related amplified polymorphisms.

The term “SCAR” as used herein refers to sequence-characterized amplified region nucleotide molecules useful as markers.

### BRIEF DESCRIPTION OF THE DRAWINGS

The present invention will be described in conjunction with reference to the following drawings, in which:

FIG. 1 is chart showing the susceptability of selected Brassica napus and B. juncea cultivars to Leptosphaeria maculans pathogenicity groups PG2, PG3, and PG4. Disease rates for each cultivar are mean values from 12 seedlings.

FIG. 2 is chart showing segregation of resistance in Brassica napus F₂ ‘Westar’×‘Dunkeld’ progeny against Leptosphaeria maculans PG3. The disease ratings for ‘Glacier’, ‘Quinta’, and the parents ‘Westar’ and ‘Dunkeld’ are mean values from 12 seedlings. Disease ratings for F₂ ‘Westar’×‘Dunkeld’ progeny (F₂WD) are average values from 94 susceptible progeny and 246 resistant ones;

FIG. 3 is a picture of multiple DNA gel lanes showing segregation of SRAP markers linked to resistance against Leptosphaeria maculans in F₂ ‘Westar’×‘Dunkeld’ progeny. Lanes 1-7: susceptible progeny, 7-14: resistant progeny. The four markers shown on the picture segregated within 92 F₂ ‘Westar’×‘Dunkeld’ progeny in a ratio not significantly different from 3:1. Markers BG₂₀SA₁₂₋₄₈₀ and BG₂₀SA₁₂₋₄₇₅ were absent in all susceptible progeny (lanes 1-7) and present in all resistant ones (lanes 8-14). Markers BG₂₀SA₁₂₋₄₆₅ and BG₂₀SA₁₂₋₄₆₀ were present in 67 progeny, including all the 23 susceptible ones, and absent in the remaining 25 resistant ones, such as in lane 10;

FIG. 4 is a picture of gel segregation of the SCAR marker BN204 linked to resistance against Leptosphaeria maculans PG3 in F2 Westar×Dunkeld progeny. M: 100 bp ladder, W: ‘Westar’, D: ‘Dunkeld’, and 1-12: F₂ Westar'×‘Dunkeld’ progeny. 1-3 and 9 represent homozygote susceptible progeny, 4, 5, and 7 are homozygote resistant progeny, and 6, 8 as well as 10-12 are heterozygote resistant progeny;

FIG. 5 is map showing the linkage group of ‘Westar’×‘Dunkeld’ population with marker loci linked to resistance gene against Leptosphaeria maculans PG3 from ‘Dunkeld’ (Rpg3Dun). Genetic distance values are expressed in cM units;

FIG. 6 is a sequence listing for a first SCAR marker referred to herein as BN204A (SEQ ID NO: 1) complementary to the Rpg3Dun gene sequence and accessible with GenBank® accession number EU122190; and

FIG. 7 is a sequence listing for a second SCAR marker referred to herein as BN204B (SEQ ID NO: 2) complementary to the Rpg3Dun gene sequence and accessible with GenBank° accession number EU122191.

### DETAILED DESCRIPTION OF THE INVENTION

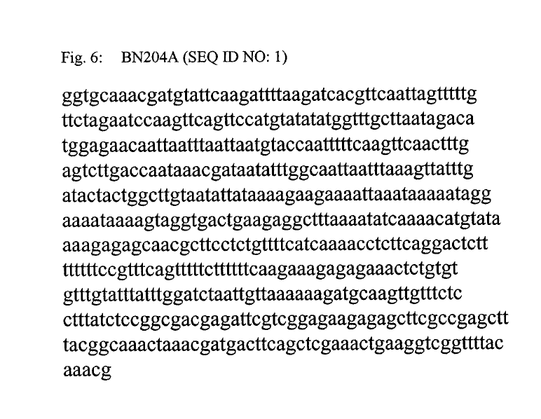

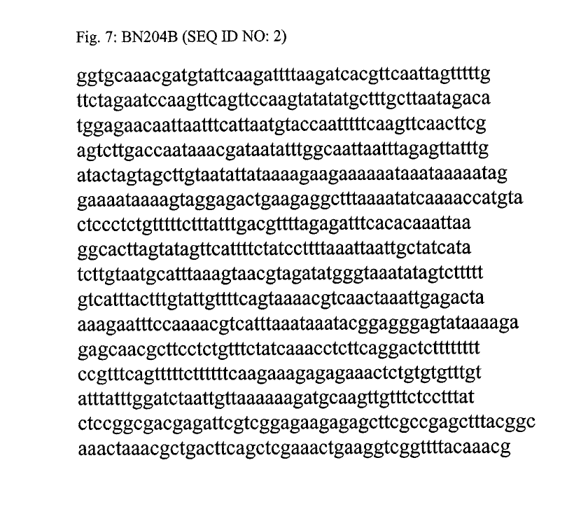

The present invention is directed to a first nucleic acid molecule having the nucleotide sequence set forth in SEQ ID NO: 1 (FIG. 6) and named “BN204A”, and a second nucleic acid molecule set forth in SEQ ID NO:2 (FIG. 7) and named “BN204B”. BN204A and BN204B are configured to be complementary to a putative gene sequence “Rpg₃Dun” occasionally present in Brassica cultivars, to methods for preparation of BN204A and BN204B, to nucleotide cassettes comprising at least one of BN204A and BN204B, to methods for preparing nucleotide cassettes comprising at least one of BN204A and BN204B, to screening methods for the use of at least one of BN204A and BN204B to identify progeny individuals from Brassica breeding programs that contain therein putative gene sequence Rpg₃Dun, to kits comprising at least one of BN204A and BN204B for using in Brassica progeny screening programs.

We have determined that the presence of putative gene sequence Rpg₃Dun, in the genome of a Brassica cultivar is highly associated with the cultivar's resistance to infection by L. maculans PG3. Because of the recent rapid spread of the occurrence of canola blackleg disease caused by L. maculans PG3, it is desirable to incorporate L. maculans PG3-resistant cultivars comprising the putative gene sequence Rpg₃Dun, into canola breeding programs. We have created SCAR markers exemplified by nucleic acid molecules BN204A and BN204B, and have determined that they are closely linked to the putative gene sequence Rpg₃Dun, as disclosed in more detail below.

### Example 1

Search of sources of resistance. A growth chamber experiment repeated once was conducted with entries from the University of Manitoba Canola and Brassica Germplasm Bank to screen for resistance against isolates of L. maculans PG2, PG3, and PG4 using the cotyledon inoculation and symptom assessment procedures as previously described by Chen at al. (2006, Can. J. Plant Pathol. 28: 533-539) and Williams (1985, Crucifer Genetic Cooperatives (CrGC) Resource Book. University of Wisconsin, Madison, WS, USA). Differential canola cultivars Westar, Glacier, and Quinta were used as references. Cultivars screened for resistance included B. napus ‘Crésor’, ‘Dunkeld’, ‘Oscar’, ‘Rainbow’, and ‘Surpass 400’, as well as B. juncea ‘Cutlass’ and ‘Dorno’.

Mapping populations. Based on results from the resistance-screening experiment, cultivar Dunkeld was used as a resistant parent in reciprocal crosses with the susceptible cultivar Westar. F₁ progeny from a ‘Westar’ female parent were evaluated for cotyledon resistance and self-pollinated for the production of F₂ progeny. Segregation of resistance against blackleg caused by L. maculans PG3 in the F₂ generation was analyzed at the cotyledon stage, with ‘Westar’, ‘Glacier’, and ‘Quinta’ being included as references. Progeny that exhibited a severity rating of 5 or less on a 0-9 rating scale were considered resistant while those with a higher score were considered susceptible.

DNA isolation and SRAP assays. Leaf and stem tissues from 340 F₂ progeny were collected, freeze dried, ground, and used for genomic DNA isolation with a salt buffer based extraction protocol (Aljanabi et al., 1997, Nucleic Acids Res. 25: 4692-4693). Bulked segregant analysis (BSA) as described by Michelmore et al. (1991, Proc. Natl. Acad. Sci. USA 88:9828-9832), was used together with sequence-related amplified polymorphisms (SRAP) as described by Li et al., (2001, Theor. Appl. Genet. 103:455-461), to search for markers polymorphic among the progeny and to identify those canola resistance against L. maculans PG3 from ‘Dunkeld’. A 22-sample set of genomic DNA from ‘Westar’ and ‘Dunkeld’ parents, 10 susceptible progeny, and 10 resistant progeny, along with 2 bulk samples, one from resistant and the other from susceptible progeny, were used for screening 180 primer combinations. Ten primer sets that had amplified markers that were polymorphic between parents and among the progeny samples were used to analyze marker segregation within a 92-progeny mapping population. These primer sets, listed in Table 1, are: BG₁₆/ODD₃ (SEQ ID NO: 3), BG₁₈/ODD₂₀ (SEQ ID NO: 4), BG₂₀/SA₁₂ (SEQ ID NO: 5), EM₂/ME₂ (SEQ ID NO: 7), ME₂/DC₁ (SEQ ID NO: 8), Na₁₂A₀₂F/Na₁₂A₀₂R (SEQ ID NO: 9), NA₁₂D₀₄F/NA₁₂D₀₄R (SEQ ID NO: 11), Na₁₂F₁₂F/Na₁₂F₁₂R (SEQ ID NO: 13), NGA₁₁₁F/NGA₁₁₁R (SEQ ID NO: 15), PM111/ODD₃ (SEQ ID NO: 18). The segregation of 2 SRAP markers amplified by Na₁₂A₀₂F/Na₁₂A₀₂R primer set that were linked to resistance from ‘Dunkeld’ was confirmed in an additional 248-progeny sample set. PCR amplification reactions were performed in a 13-μL reaction volume containing 15 ng template DNA, 0.4 μM for each of two primers, 0.75 units of Taq polymerase (Fisher brand), 100 mM Tris-HCl (pH 8.0), 500 mM KCl, 1.5 mM MgCl₂, and 0.1 mM each of dNTPs. PCR amplification was run in a programmable thermal controller (Eppendorf® MasterCycler® Gradient, Eppendorf Canada Ltd., Mississauga, ON. Canada; Eppendorf and MasterCycler are registered trademarks of Eppendorf AG, Hamburg, Fed. Rep. Germany). The first 5 cycles were run at 94° C. for 1 min, 35° C. for 50 sec, and 72° C. for 1 min for denaturing, annealing and extension, respectively. Then, the remainder of the amplification was 36 cycles at 94° C. for 50 sec, 50° C. for 50 sec and 72° C. for 1 min. PCR products were separated by electrophoresis using a denaturing 5% polyacrylamide gel containing 7.5M urea. Gels were then silver-stained with the Promega® kit (Promega Corp., Madison, Wis., USA; Promega is a registered trademark of the Promega Corp.) according to the manufacturer's specifications. PCR assays were replicated once in order to confirm observed markers. The presence and absence of all fragments between molecular sizes of 50 and 500 base pairs (bp) were scored for each sample. Bands larger than 500 bp or less than 50 bp were not scored because of the insufficient resolution.

DNA sequencing and SCAR development. DNA bands of SRAP markers linked to resistance were collected from polyacrylamide gels, ground in 50 μL of TE and incubated overnight at 4° C. in order to isolate DNA fragments. These fragments were used as template for PCR assays with the corresponding original SRAP primers. The PCR mixture composition used was similar to the one used in the SRAP assays except that cycling protocol was as follows: initial denaturation at 95° C. for 5 min; 35 cycles at 94° C. for 30 sec, 55° C. for 20 sec and 72° C. for 1 min; and a final DNA extension at 72° C. for 6 min. PCR products were purified using the Qiaquick® PCR purification kit (Qiaquick is a registered trademark of Qiagen GMBH Corp., Hilden Fed. Rep. Germany) according to the producer specifications (Qiagen Inc., Mississauga, ON. Canada). Purified PCR products were sent for sequencing at Macrogen® DNA Sequencing Service (Macrogen USA, Rockville, Md.; Macrogen is a registered trademark of Oxford BioMedica PLC Corp., Oxford, Great Britain). The software ClustalX was used as suggested by Thompson et al. (1997, Nucl. Acids Res 24:4876-4882) for sequence alignment, while sequence alignments were edited with the GeneDoc® software (GeneDoc is a registered trademark owned by Karl B. Nicholas, San Raphael, Calif., USA) as outlined by Nicholas et al. (1997, EMBNEW.NEWS 4:14). Similarity searching with public library sequences was performed with BLASTn following the method of Altschul et al (1990, J. Mol. Biol. 215:403-10). A Genbank library sequence with a genomic region similar to our query was used to design new primers in order to develop sequence characterized amplified region (SCAR) markers linked to resistance in ‘Dunkeld’ against L. maculans PG3. These new primers were used to amplify genomic DNA from ‘Westar’ and ‘Dunkeld’. The segregation of polymorphic markers was then analyzed with the 22-progeny sample set used for screening of SRAP primer combinations. Primer sets with markers that segregated as expected were used in analyzing the 92-progeny sample set for linkage mapping. The markers were later confirmed using the additional 248-progeny sample set.

Data analysis. Chi-square test was run with MS Excel software (Excel is a registered trademark of the Microsoft Corp., Redmond, Wash., USA) to verify if segregation ratios of the resistance trait and molecular markers fit Mendelian ratios. Linkage analysis was performed with MapMaker Exp 3.0 computer program following methods taught by Lander et al. (1987, Genomics 1:174-181) and Lincoln et al., (1992, Whitehead Institute Technical Report. 3rd Ed.) in order to identify molecular markers linked to the ‘Dunkeld’ resistance gene against blackleg caused by L. maculans PG3.

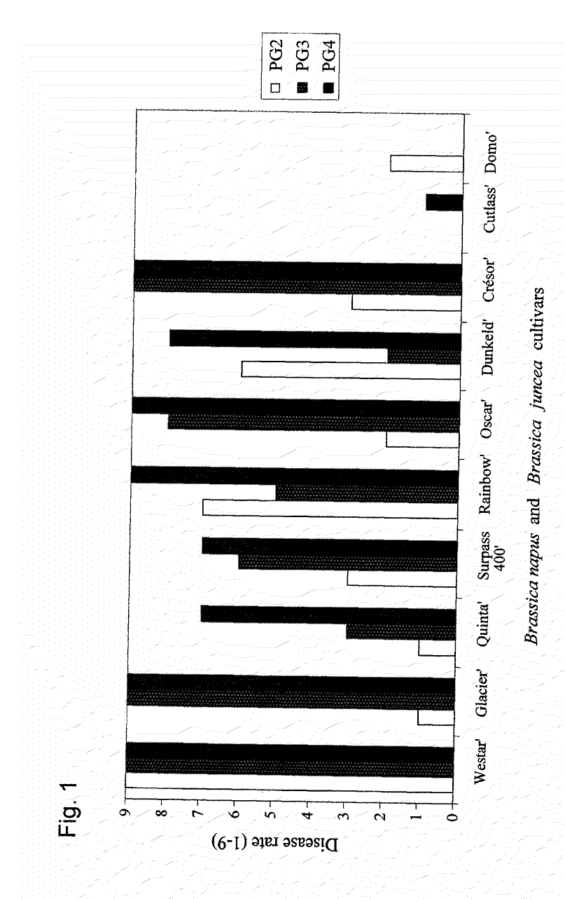

Results. The winter cultivar ‘Quinta’ used as a differential in L. maculans pathogenicity group testing is known to carry cotyledon stage resistance genes against L. maculans PG2 and PG3. We found a comparable level of resistance against PG3 in the Australian spring canola cultivar Dunkeld. B. juncea cultivars Cutlass and Domo exhibited resistance against PG2, PG3, and PG4 (FIG. 1).

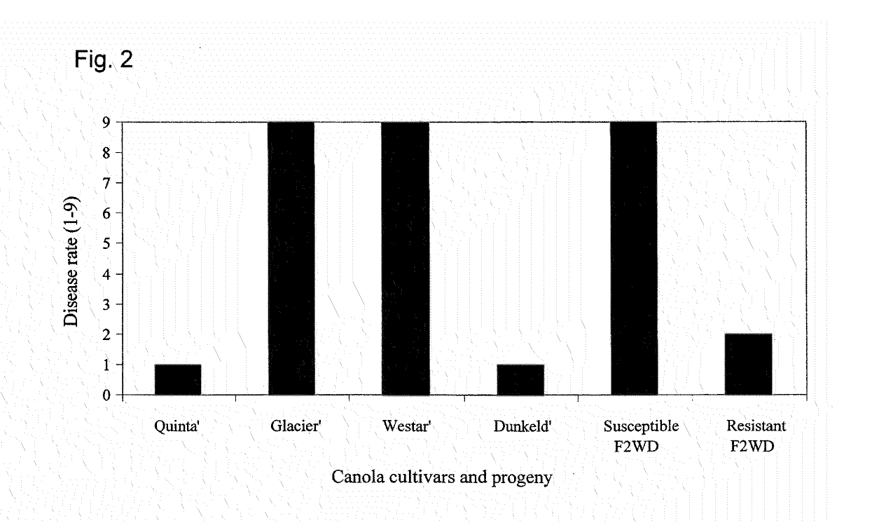

Tests of 340 F₂ ‘Westar’×‘Dunkeld’ progeny for response to PG3 revealed that 94 progeny were susceptible and 246 were resistant. Disease symptom ratings, on a 0-9 scale, ranged from 1 to 5 for resistant progeny, with an overall average rating of 2, while the rating for all susceptible progeny was 9 (FIG. 2). According to chi-square analysis (χ²=0.96), the segregation ratio of the resistance against L. maculans PG3 within F₂ ‘Westar’×‘Dunkeld’ progeny was not significantly different from 3:1, which was consistent with a single dominant gene model.

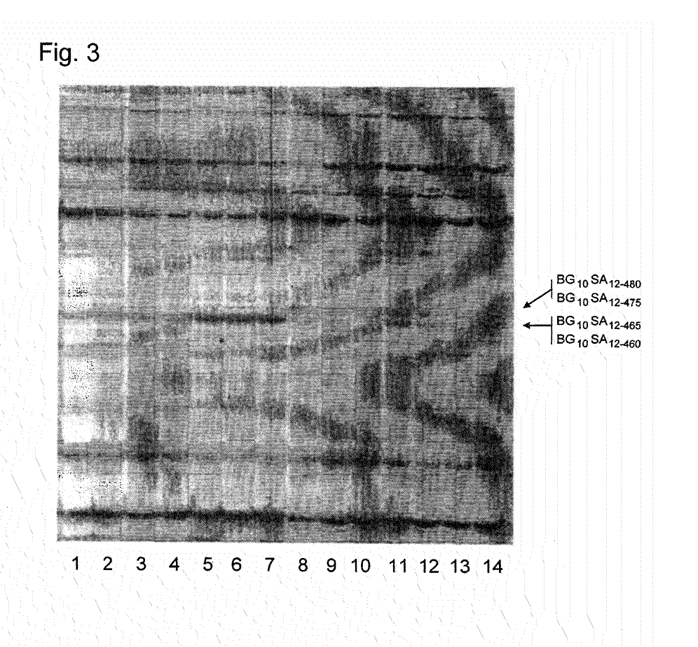

Bulked segregant analysis (BSA) used together with the SRAP methods allowed to select from 180 primer combinations 10 primer pairs (Table 1) that produced polymorphic markers between parents and within progeny. 52 polymorphic markers within 92 F₂ ‘Westar’×‘Dunkeld’ progeny were obtained from these 10 primer combinations. The segregation of four SRAP markers including two from each of Na₁₂A₀₂F/Na₁₂A₀₂R and BG₂₀/SA₁₂ primer combinations was consistent with the resistance segregation within the 92 tested progeny. The two markers from the first primer combination were 200 bp and 190 bp and the markers from the last primer pair were 480 bp and 475 bp (FIG. 3).

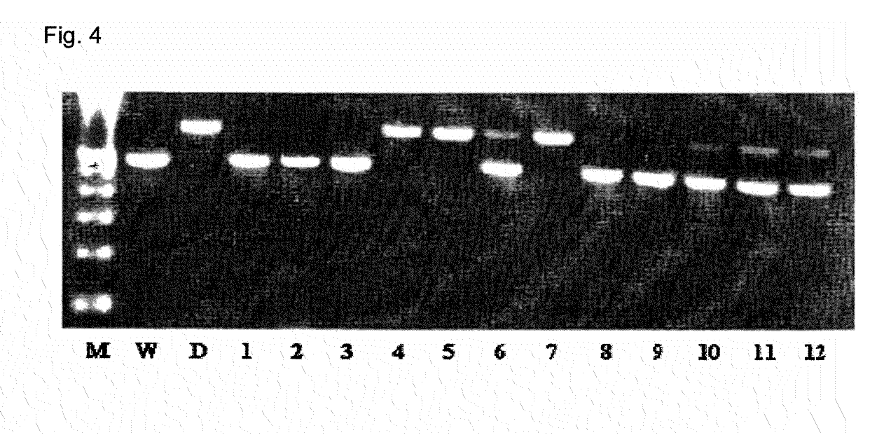

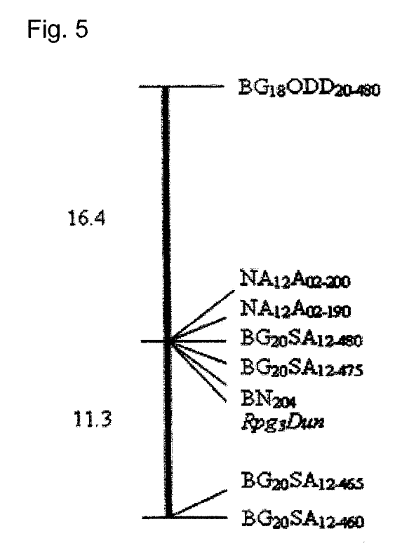

Sequence results from the two NA₁₂A₀₂ markers showed that they are from CT simple sequence repeat (SSR) regions and that the two units differentiate ‘Westar’ from ‘Dunkeld’ with 17 and 19 repeats, respectively. BLASTn analysis revealed that this sequence has 96% homology (e-value=1e⁻²⁷) with a region of the Brassica rapa subsp. pekinensis BAC clone KBr0334114 available in Genbank under the accession number AC189311.1. Forward primer BN204F and reverse primer BN204R (Table 1; SEQ ID NO: 20 and SEQ ID NO: 21 respectively) were designed from that clone sequence and provided SCAR markers that are also linked to the variety Dunkeld resistance against L. maculans. The segregation ratio of the BN204 markers within the F₂ ‘Westar’×‘Dunkeld’ was consistent with a single dominant gene model and also allowed differentiation of homozygote from heterozygote resistant progeny (FIG. 4). Linkage analysis identified a linkage group on which the ‘Dunkeld’ resistance gene (Rpg₃Dun) against L. maculans PG3 was closely linked to the two BN204 SCAR markers and four SRAP markers (FIG. 5).

Three additional SRAP markers including two from BG₂₀/SA₁₂ and one from BG₁₈/ODD₂₀ were located at 11.3 and 16.4 cM respectively each side of the core region with the resistance gene. The number of progeny recombinant for the resistance trait and the molecular markers analyzed in 340 progeny, the SRAP marker NA₁₂A₀₂₋₂₀₀ and the SCAR marker BN204, was not significant (Table 2).

The screening of the University of Manitoba Brassica germplasm revealed that sources of canola resistance against blackleg caused by L. maculans PG3 were available in two B. napus and two B. juncea varieties. We have selected ‘Dunkeld’ for further analysis because, being a spring type, it is easier to manipulate than the winter cultivar ‘Quinta’ and the B. juncea ‘Cutlass’ and ‘Domo’ in crossing with B. napus spring cultivars, such as ‘Westar’, and commercial cultivars for genetics studies and gene pyramiding. The resistance in ‘Dunkeld’ against blackleg is thought to be polygenic in part and can be traced back to possible sources present into its pedigree such as B. napus ‘Chikuzen’ and ‘Norin’ and B. juncea BJ168 (Gororo et al., 2004, Proceedings of the 4^{th} International crop Science Congress. Brisbane, Australia, Sep. 26-Oct. 1, 2004; Mailer et al., Aust. J. Exp. Agric. 37: 793-800). Our resistance screening experiment revealed that ‘Dunkeld’ was resistant against L. maculans PG3, but was susceptible to PG2 and PG4. The results suggest the presence of at least one major resistance gene in addition to polygenic resistance. These results also support the supposition that the decline of ‘Dunkeld’ blackleg resistance and reduced grain yield recorded in Australia from 1999 to 2001 was due to a change in virulence in the blackleg pathogen (Gororo et al.). As the three pathogenicity groups coexist in Australia (Balesdent, 2005, Phytopathology 92:1122-1133), the change in virulence may have resulted from the pathogen population composition shifting to higher proportions of PG2 and PG4 after the release of ‘Dunkeld’. The conclusion that a major gene known as Rpg₃Dun is responsible for resistance in ‘Dunkeld’ against PG3 was supported by the resistance segregation ratio of 3:1 in F2 ‘Westar’×‘Dunkeld’ progeny. Rpg₃Dun is likely similar to the Rlm4 gene, which was previously suggested to be the major resistance gene in ‘Dunkeld’ (Rouxel et al., 2003, Euphytica 133:219-231). Rlm4 has also been located in a cluster of four other tightly linked genes including Rlm3, Rlm7 and Rlm9 (Delourme, 2004, Phytopathology 94:578-583).

TABLE 1
Characteristics of primers used in this study

| Primer name | Primer sequence (5′-3′) | Source | SEQ ID NO: |
|---|---|---|---|
| BG₁₆ | TGATACCACTTGCGATACCA | Dr. G. Li¹ | 3 |
| BG₁₈ | GCAAGTCTCTCAGGTTATTC | Dr. G. Li | 4 |
| BG₂₀ | TCCTCTCCACTTTTGTCTTC | Dr. G. Li | 5 |
| DC₁ | TAAACAATGGCTACTCAAG | Dr. G. Li | 6 |
| EM₂ | GACTGCGTACGAATTCTG C | Dr. G. Li | 7 |
| ME₂ | TGAGTCCAAACCGGAGC | Dr. G. Li | 8 |
| Na₁₂A₀₂F | AGCCTTGTTGCTTTTCAACG | Dr. M. Trick² | 9 |
| Na₁₂A₀₂R | AGTGAATCGATGATCTCGCC | Dr. M. Trick | 10 |
| Na₁₂D₀₄F | ACGGAGTGATGATGGGTCTC | Dr. M. Trick | 11 |
| Na₁₂D₀₄R | CCTCAATGAAACTGAAATATGTGTG | Dr. M. Trick | 12 |
| Na₁₂F₁₂P | CGTTCTCACCTCCGATAAGC | Dr. M. Trick | 13 |
| Na₁₂F₁₂R | TCCGATGTAGAATCAGCAGC | Dr. M. Trick | 14 |
| NGA₁₁₁F | TGTTTTTTAGGACAAATGGCG | Dr. M. Trick | 15 |
| NGA₁₁₁R | CTCCAGTTGGAAGCTAAAGGG | Dr. M. Trick | 16 |
| ODD₃ | CCAAAACCTAAAACCAGGA | Dr. G. Li | 17 |
| PM₁₁₁ | CTTTAGCAGCATTGTTACCA | Dr. G. Li | 18 |
| SA₁₂ | TTCTAGGTAATCCAACAACA | Dr. G. Li | 19 |
| BN204F | GGTGCAAACGATGTATTCAAGA | This study | 20 |
| BN204R | CGTTTGTAAAACCGACCTTCA | This study | 21 |

¹Dr G. Li, University of Manitoba, Winnipeg, Manitoba, Canada
²Dr M. Trick, Brassica DB, John Imes Centre, Norwich, UK

TABLE 2
Segregation of markers linked to the
cultivar ‘Dunkeld’ resistance against
L. maculans PG3 within 340 F₂
‘Westar’ × ‘Dunkeld’ progeny

| Marker | χ² |
|---|---|
| Rpg₃Dun* | 1.56 |
| Na₁₂A₀₂₋₂₀₀ | 0.06 |
| BN204 | 0.25 |

*Resistance testing results

Molecular markers linked to the ‘Dunkeld’ resistance gene against L. maculans PG3 were identified with a combination of BSA and SRAP procedures. A linkage group with eight molecular markers linked to this resistance trait was identified. DNA sequencing and BLAST analysis of a sequence from one of these markers allowed us to physically locate this resistance gene on a BAC clone from Brassica rapa subsp. pekinensis. BLASTx results also revealed that these markers are located close to a genomic region homologous to the Arabidopsis region encoding a serine/threonine ste20-like kinase. Protein kinases have several functions including defense responses (Hardie, 1999, Annu. Rev. Plant Physiol. Plant Mol. Biol. 50: 97-131). Plant serine/threonine kinases and kinase receptors involved in disease resistance have been reported in crops such as barley, tomato and rice (Brueggeman et al., 2006, Theor appl Genet 113:1147-1158; Liu et al., 2002, J. Biol. Chem 277:20264-20269; Loh et al., 1995, Plant Physiol 108:1735-1739; Nirmala et al., 2006, Proc. Natl. Acad. Sci. USA 103:7518-7523). Results from linkage mapping and sequence analysis suggested that the ‘Dunkeld’ resistance trait against L. maculans PG3 might be associated with a gene encoding protein kinase. The gene analysis revealed that it contains open reading frames producing different putative transcripts that could be alternatively expressed in different stress conditions (9, 36). The SCAR marker BN204 linked to Rpg3Dun resistance gene can be used in marker-assisted selection of canola resistance against blackleg caused by L. maculans PG3.

In view of numerous changes and variations that will be apparent to persons skilled in these arts, the scope of the present invention is to be considered limited solely by the appended claims.

1. An isolated nucleic acid molecule comprising a nucleotide sequence set forth in SEQ ID NO: 1, said nucleotide sequence complementary to a Rpg3Dun gene sequence.

2. An isolated nucleic acid molecule comprising a nucleotide sequence set forth in SEQ ID NO: 2, said nucleotide sequence complementary to a Rpg3Dun gene sequence.

3. An isolated nucleic acid molecule comprising a nucleotide sequence set forth in SEQ ID NO: 20, said nucleotide sequence complementary to a Rpg3Dun gene sequence.

4. An isolated nucleic acid molecule comprising a nucleotide sequence set forth in SEQ ID NO: 21, said nucleotide sequence complementary to a Rpg3Dun gene sequence.

5. A method for screening a plurality of plant cultivars for identification of individual cultivars resistant to infection by Leptosphaeria maculans, said method comprising the steps of:
collecting from each individual cultivar comprising the plurality of cultivars, a biomass sample;
preparing a nucleic acid preparation from each of said biomass samples;
contacting each of said nucleic acid preparations with at least one of a nucleic acid molecule comprising a nucleotide sequence set forth in SEQ ID NO: 1, a nucleic acid molecule comprising a nucleotide sequence set forth in SEQ ID NO: 2, a nucleic acid molecule comprising a nucleotide sequence set forth in SEQ ID NO: 20, and a nucleic acid molecule comprising a nucleotide sequence set forth in SEQ ID NO: 21, and intermixing therewith;
conducting an amplification reaction therewith each of said intermixed nucleic acid preparations; and
assessing each of said amplified nucleic acid preparations to identify individual nucleic acid preparations wherein a hybridization reaction with one of said nucleotide sequences occurred during the amplication reaction, wherein a hybridization reaction is indicative of the cultivar's resistance to infection by Leptosphaeria maculans.

6. A method according to claim 5, wherein the plant cultivars are Brassica plant cultivars.

7. A method for screening a plurality of plant cultivars for identification of individual cultivars containing therein Rpg3Dun gene sequences, said method comprising the steps of:
collecting from each individual cultivar comprising the plurality of cultivars, a biomass sample;
preparing a nucleic acid preparation from each of said biomass samples;
contacting each of said nucleic acid preparations with at least one of a nucleic acid molecule comprising a nucleotide sequence set forth in SEQ ID NO: 1, a nucleic acid molecule comprising a nucleotide sequence set forth in SEQ ID NO: 2, a nucleic acid molecule comprising a nucleotide sequence set forth in SEQ ID NO: 20, and a nucleic acid molecule comprising a nucleotide sequence set forth in SEQ ID NO: 21, and intermixing therewith;
conducting an amplification reaction therewith each of said intermixed nucleic acid preparations; and
assessing each of said amplified nucleic acid preparations to identify individual nucleic acid preparations wherein a hybridization reaction with one of said nucleotide sequences occurred during the amplication reaction, wherein a hybridization reaction is indicative of the cultivar's containing therein Rpg3Dun gene sequences.

8. A method according to claim 7, wherein the plant cultivars are Brassica plant cultivars.

9. A method for generating or increasing the resistance of Brassica plants to infection by Leptosphaeria maculans pathogenicity group PG3, the method comprising the steps of:
using the method of claim 8 to select a Brassica plant cultivar containing therein Rpg3Dun genes;
including the selected Brassica plant cultivar in a Brassica breeding program for resistance to Leptosphaeria maculans pathogenicity group PG3, and producing progeny therefrom;
screening the progeny with the method of claim 8 to further select Brassica plant cultivars containing therein the Rpg3Dun gene; and
challenging the further selected Brassica plant cultivars with Leptosphaeria maculans pathogenicity group PG3, to confirm resistance of the further selected Brassica plant cultivars to infection by Leptosphaeria maculans pathogenicity group PG3.

10. A gene cassette comprising at least one nucleotide sequence selected from the group consisting of a nucleotide sequence set forth in SEQ ID NO: 2, a nucleotide sequence set forth in SEQ ID NO: 2, a nucleic acid molecule comprising a nucleotide sequence set forth in SEQ ID NO: 20, and a nucleic acid molecule comprising a nucleotide sequence set forth in SEQ ID NO: 21.

11. A kit comprising a nucleotide sequence selected from the group consisting of a nucleotide sequence set forth in SEQ ID NO: 1, a nucleic acid molecule comprising a nucleotide sequence set forth in SEQ ID NO: 2, a nucleic acid molecule comprising a nucleotide sequence set forth in SEQ ID NO: 20, and a nucleic acid molecule comprising a nucleotide sequence set forth in SEQ ID NO: 21, said nucleotide sequence complementary to a Rpg3Dun gene sequence.


SEQUENCE LISTING (21 sequences)

SEQ ID NO: 1 - DNA, 555 bp, Brassica napus
ggtgcaaacgatgtattcaagattttaagatcacgttcaattagtttttgttctagaatccaagttcagttccatgtatatatggtttgcttaatagacatggagaacaattaatttaattaatgtaccaatttttcaagttcaactttgagtcttgaccaataaacgataatatttggcaattaatttaaagttatttgatactactggcttgtaatattataaaagaagaaaattaaataaaaataggaaaataaaagtaggtgactgaagaggctttaaaatatcaaaacatgtataaaagagagcaacgcttcctctgttttcatcaaaacctcttcaggactcttttttttccgtttcagtttttcttttttcaagaaagagagaaactctgtgtgtttgtatttatttggatctaattgttaaaaaagatgcaagttgtttctcctttatctccggcgacgagattcgtcggagaagagagcttcgccgagctttacggcaaactaaacgatgacttcagctcgaaactgaaggtcggttttacaaacg

SEQ ID NO: 2 - DNA, 799 bp, Brassica napus
ggtgcaaacgatgtattcaagattttaagatcacgttcaattagtttttgttctagaatccaagttcagttccaagtatatatgctttgcttaatagacatggagaacaattaatttcattaatgtaccaatttttcaagttcaacttcgagtcttgaccaataaacgataatatttggcaattaatttagagttatttgatactagtagcttgtaatattataaaagaagaaaaaataaataaaaataggaaaataaaagtaggagactgaagaggctttaaaatatcaaaaccatgtactccctctgtttttctttatttgacgttttagagatttcacacaaattaaggcacttagtatagttcattttctatccttttaaattaattgctatcatatcttgtaatgcatttaaagtaacgtagatatgggtaaatatagtctttttgtcatttactttgtattgttttcagtaaaacgtcaactaaattgagactaaaagaatttccaaaacgtcatttaaataaatacggagggagtataaaagagagcaacgcttcctctgtttctatcaaacctcttcaggactcttttttttccgtttcagtttttcttttttcaagaaagagagaaactctgtgtgtttgtatttatttggatctaattgttaaaaaagatgcaagttgtttctcctttatctccggcgacgagattcgtcggagaagagagcttcgccgagctttacggcaaactaaacgctgacttcagctcgaaactgaaggtcggttttacaaacg

SEQ ID NO: 3 - DNA, 20 bp, Artificial sequence
  Primer BG16
tgataccacttgcgatacca

SEQ ID NO: 4 - DNA, 20 bp, Artificial sequence
  Primer BG18
gcaagtctctcaggttattc

SEQ ID NO: 5 - DNA, 20 bp, Artificial sequence
  Primer BG20
tcctctccacttttgtcttc

SEQ ID NO: 6 - DNA, 19 bp, Artificial sequence
  Primer DC1
taaacaatggctactcaag

SEQ ID NO: 7 - DNA, 19 bp, Artificial sequence
  Primer EM2
gactgcgtacgaattctgc

SEQ ID NO: 8 - DNA, 17 bp, Artificial sequence
  Primer ME2
tgagtccaaaccggagc

SEQ ID NO: 9 - DNA, 20 bp, Artificial sequence
  Primer Na12A02F
agccttgttgcttttcaacg

SEQ ID NO: 10 - DNA, 20 bp, Artificial sequence
  Primer No. Na12A02R
agtgaatcgatgatctcgcc

SEQ ID NO: 11 - DNA, 20 bp, Artificial sequence
  Primer Na12D04F
acggagtgatgatgggtctc

SEQ ID NO: 12 - DNA, 25 bp, Artificial sequence
  Primer Na12D04R
cctcaatgaaactgaaatatgtgtg

SEQ ID NO: 13 - DNA, 20 bp, Artificial sequence
  Primer Na12F12F
cgttctcacctccgataagc

SEQ ID NO: 14 - DNA, 20 bp, Artificial sequence
  Primer Na12F12R
tccgatgtagaatcagcagc

SEQ ID NO: 15 - DNA, 21 bp, Artificial sequence
  Primer NGA111F
tgttttttaggacaaatggcg

SEQ ID NO: 16 - DNA, 21 bp, Artificial sequence
  Primer NGA111R
ctccagttggaagctaaaggg

SEQ ID NO: 17 - DNA, 19 bp, Artificial sequence
  Primer ODD3
ccaaaacctaaaaccagga

SEQ ID NO: 18 - DNA, 20 bp, Artificial sequence
  Primer PM111
ctttagcagcattgttacca

SEQ ID NO: 19 - DNA, 20 bp, Artificial sequence
  Primer SA12
ttctaggtaatccaacaaca

SEQ ID NO: 20 - DNA, 22 bp, Artificial sequence
  Primer BN204F
ggtgcaaacgatgtattcaaga

SEQ ID NO: 21 - DNA, 21 bp, Artificial sequence
  Primer BN204R
cgtttgtaaaaccgaccttca